(19) United States
(12) Patent Application Publication
(10) Pub. No.: US 2023/0347260 A1
(43) Pub. Date: Nov. 2, 2023

(54) Selecting Resins for Use in Chromatography Purification Processes

(71) Applicant: AMGEN INC., Thousand Oaks, CA (US)

(72) Inventors: Hector Nogueras, Cayey, PR (US); Myra Coufal, Quincy, MA (US); Deborah Soto-Ortega, Caguas, PR (US)

(21) Appl. No.: 18/007,126

(22) PCT Filed: Jul. 16, 2021
(86) PCT No.: PCT/US21/41973

§ 371 Date: Jan. 27, 2023

Related U.S. Application Data
(60) Provisional application No. 63/058,050, filed on Jul. 29, 2020.

(51) Int. Cl. B01D 15/10; G16C 20/30; C07K 1/18

(52) U.S. Cl. CPC B01D 15/10; G16C 20/30; C07K 1/18

(57) ABSTRACT

A method of selecting raw materials for use in a column chromatography purification process in which, for each of one or more candidate resins, a respective set of resin attribute values is received, including at least one analytical measurement of the candidate resin. The method also includes, for each candidate resin, predicting a respective value of a performance indicator for the column chromatography purification process by applying the respective set of resin attribute values, and possibly other parameter values (e.g., harvest filtrate and/or purification process parameters) as inputs to a multivariate statistical model. The method further includes selecting a resin of the one or more candidate resins based at least in part on the predicted respective value(s) of the performance indicator, and performing the column chromatography purification process using the selected resin as a stationary phase.

### FIELD OF DISCLOSURE

The present disclosure relates generally to the production of biopharmaceutical products, and more specifically to techniques for facilitating the selection (e.g., screening) of resins for column chromatography purification processes in a manner that accounts for variability (e.g., lot-to-lot variability) in resin attributes.

### BACKGROUND

In the biopharmaceutical industry, large, complex protein molecules known as biologics or therapeutic proteins are derived from living systems. At a high level, the process of manufacturing the therapeutic proteins includes the following stages: (1) a host cell selection stage, in which the master cell line containing the gene that makes the desired protein is produced (e.g., using Chinese hamster ovary (CHO) cells); (2) a cell culture stage, in which defined culture media are used to grow large numbers of cells that produce the protein in bioreactors; (3) a purification stage, in which the recovery and purification of the product from the previous stage is performed to isolate the protein; and (4) a formulation and fill-finish-package stage, in which the protein is prepared for use by physicians or patients.

FIG. 2 depicts a typical purification process 10 for manufacturing therapeutic proteins (i.e., the third stage listed above). At a first step 12, the therapeutic protein (also referred to as the “target molecule”) is harvested from the cell culture (e.g., from bioreactors). The harvested material also contains undesired by-products, such as host cell proteins (HCP) that were secreted along with the target molecule and/or released into the process stream during the harvesting steps, as well as other undesired matter (e.g., degraded or aggregated proteins). At step 14, the harvested material undergoes purification via column chromatography, typically using multiple chromatography columns. The purification at step 14 plays a major role in removing HCP and other impurities from the harvested material. As one example, step 14 may include purification via four chromatography columns, with a first column being designed to select the target molecule and reduce host cell impurities, the second column reducing both product-related and process-related impurities, the third column further concentrating the desired product, and the fourth column further increasing the product purity. Thereafter, at steps 16 and 18, the purified substance undergoes viral filtration and final filtration, respectively. The purified/filtered substance is then typically formulated with an excipient to produce a sterile solution that can be injected or infused, and placed in a target buffer, yielding a formulation that is placed within containers (e.g., vials or syringes) for labeling, long-term storage, and shipment. While the example purification process 10 is provided for illustrative purposes, it will be appreciated that the techniques described herein can readily be applied to other purification processes that include column chromatography.

In general, “chromatography” (e.g., as performed at step 14) refers to a separation process wherein molecules are distributed between two phases: (1) a stationary phase, which is often a resin; and (2) a mobile phase, which in the case of protein separation is a solvent, such as water or chloroform. Molecules that are more strongly attracted to the stationary phase move more slowly through the system as compared to those that are more strongly attracted to the mobile phase. For commercial manufacturing purification, chromatography is typically carried out as column chromatography due to scale considerations. In a common chromatographic operation, a sample volume is injected into the column. Eluent is then pumped through the column, causing molecules to be separated based on their relative affinity for the stationary resin and the eluent. Different molecules will elute from the column at different times and after different volumes of eluent have passed through the column. Accordingly, therapeutic proteins can be separated from other substances that elute from the column at times earlier or later than the therapeutic proteins. This information is captured in a chromatogram, which is a plot, e.g., a UV absorbance plot, of the concentration exiting the column versus time.

To provide a few examples: hydrophobic interaction chromatography can be used to separate proteins based on differences in hydrophobicity, affinity chromatography can be used to separate molecules based on differences in affinity for a target ligand attached to a chromatography resin, and ion exchange chromatography can be used to separate molecules based on differences in molecular charge. As a more specific example, cation-exchange chromatography (CEX) is an ion exchange chromatography used when the molecule of interest is positively charged. Other common types of chromatography include size-exclusion chromatography (SEC), in which molecules in solution are separated by size and/or molecular weight, and Protein A chromatography.

In general, to ensure a robust commercial manufacturing process, it is important to characterize biological processes by, among other things, identifying how variability in the attributes of raw materials contributes to process performance and product quality. For various reasons, however, manufacturers/suppliers typically do not evaluate how raw materials manufactured at the edge of a certificate of analysis (or “CoA”), due to variability between manufacturing lots, can influence process consistency and product quality. Instead, the effects of raw material variability are evaluated, if at all, at target ranges using various risk-based analyses. Risk-based approaches of this sort may be insufficient for certain raw materials, such as the resin used as the stationary phase in column chromatography purification processes (e.g., the purification processes discussed above with reference to step 14 of FIG. 2). In particular, lot-to-lot variability of these raw materials can result in costly failure events, such as unacceptable purification performance indicators (e.g., excessive HCP levels) that require the discarding, or additional processing, of drug batches.

### SUMMARY

Embodiments described herein relate to systems and methods that facilitate the selection of a resin for the stationary phase of a column chromatography purification process when manufacturing a therapeutic protein, such as a monoclonal antibody (“mAb”), or a bispecific or other multi-specific antibody, for example. In these embodiments, a multivariate statistical model enables the selection of resins (e.g., the selection of specific resin lots) that will not degrade (or overly degrade) performance of the purification process, by accounting for variability (e.g., lot-to-lot variation) in resin manufacturing. The multivariate statistical model predicts a performance indicator, such as a level of HCP and/or one or more other impurities, for a column chromatography purification process (e.g., a CEX, SEC, Protein A, or other suitable chromatography process that uses a resin as the stationary phase), based on various resin attributes and possibly one or more other types of inputs (e.g., harvest filtrate loading material factors and/or chromatography process parameters). The resin attributes may be provided by the manufacturer within a Certificate of Analysis (CoA), for example.

Resin “selection” may refer to selecting one or more resins out of multiple candidate resins, or confirming whether a single candidate resin is acceptable for use (i.e., “screening” the candidate resin prior to commercial-scale use). For example, resin lots received from a supplier may be screened using the multivariate statistical model and CoA data provided by the manufacturer, to determine which lots are acceptable and which lots should be rejected/replaced (or will necessitate further purification steps to meet requirements, etc.). As another example, specific resin lots may be selected (e.g., ordered) in the first instance based on which lots provide the most clearance relative to acceptability thresholds (e.g., by choosing the resin lots for which the multivariate statistical model predicts the lowest HCP levels). As still other examples, resins from different manufacturers, and/or different types or formulations of resins, may be selected by applying different, corresponding sets of resin attribute values to the multivariate statistical model.

Using techniques such as these, the amount of drug substance that must be rejected/discarded, and/or the amount of time and other resources needed to ensure acceptable purification performance, may be substantially reduced. For example, the time required to ensure acceptable purification performance may be reduced from tens or even hundreds of hours down to something on the order of one or two hours.

Moreover, some embodiments described herein identify which resin attributes have the greatest effect on performance (e.g., as measured by HCP reduction) of the column chromatography purification process. These resin attributes may be identified using a small-scale model of a commercial-scale column chromatography process. When used herein as a descriptor for a particular process, the term “commercial-scale” or “commercial scale” indicates that the process is used in the course of manufacturing or testing—or in the course of identifying, obtaining and/or screening specific supplies/materials to be used in the manufacture or test of—a lot or batch of drug product that is intended for sale and/or distribution to customers (e.g., patients, pharmacies, etc.), possibly subject to one or more downstream screening steps (e.g., visual inspection of a vial or syringe filled with the manufactured drug product, etc.). Commercial scale can mean the use of bioreactors of at least 500 L, 1000 L, 2000 L or more. Conversely, “small-scale” or “small scale” indicates that a process is not commercial-scale (i.e., is performed “offline”). For example, a lab-based chromatography station for resin lot screening is “small-scale” rather than “commercial-scale” if the station is not used to screen resins lots specifically for use in commercial drug production, regardless of the physical size of the lab-based station relative to a commercial-scale chromatography station. Once the most important resin attributes have been identified, specific values or value ranges for those attributes that substantially improve purification performance are identified. The results can then be provided to the resin manufacturer, which can use the results to make appropriate changes to the resin manufacturing process. Moreover, due to the ability of the small-scale model to closely replicate commercial-scale performance, data from the small-scale model runs (e.g., resin attribute values, purification process parameters, resulting HCP levels, etc.) can be used to expand the size of the training data set for a multivariate statistical model, thereby increasing accuracy of the model. Further still, the multivariate statistical model may produce metrics that shed additional light on which resin attributes (and/or other factors) are more predictive of purification performance.

### BRIEF DESCRIPTION OF THE DRAWINGS

The skilled artisan will understand that the figures, described herein, are included for purposes of illustration and do not limit the present disclosure. The drawings are not necessarily to scale, and emphasis is instead placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a simplified block diagram of an example system that may implement the techniques described herein.

FIG. 2 depicts a prior art purification process for manufacturing therapeutic proteins.

FIG. 3 depicts measured lot-to-lot variability in commercial-scale purification performance across different resin lots.

FIG. 4 depicts example purification performance versus column height in a small-scale model of a commercial-scale column chromatography system.

FIG. 5 depicts charts that compare performance of a small-scale model to performance of a commercial-scale column chromatography system.

FIG. 6 depicts purification performance resulting from different resin lots, according to a small-scale model of a commercial-scale column chromatography system.

FIG. 7 depicts overall variability in post-purification HCP levels due to resin variability and operational variability.

FIG. 8 depicts an actual-by-predicted plot that compares performance of the model against experimental determination.

FIGS. 9A and 9B depict the effect of certain resin attributes and resin manufacturing operating parameters on purification performance according to a small-scale model of a commercial-scale column chromatography system.

FIG. 10 depicts the effect of certain resin attributes and resin manufacturing operating parameters on purification performance according to a small-scale model.

FIG. 11 depicts an example resin manufacturing process in which feedback is provided based on small-scale modeling results.

FIG. 12 depicts example modifications to the resin manufacturing process that may improve purification performance.

FIG. 13 depicts a chart that compares actual purification performance with confidence limits predicted by a multivariate statistical model.

FIG. 14 is a flow diagram depicting an example method of selecting raw materials for use in a column chromatography purification process.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

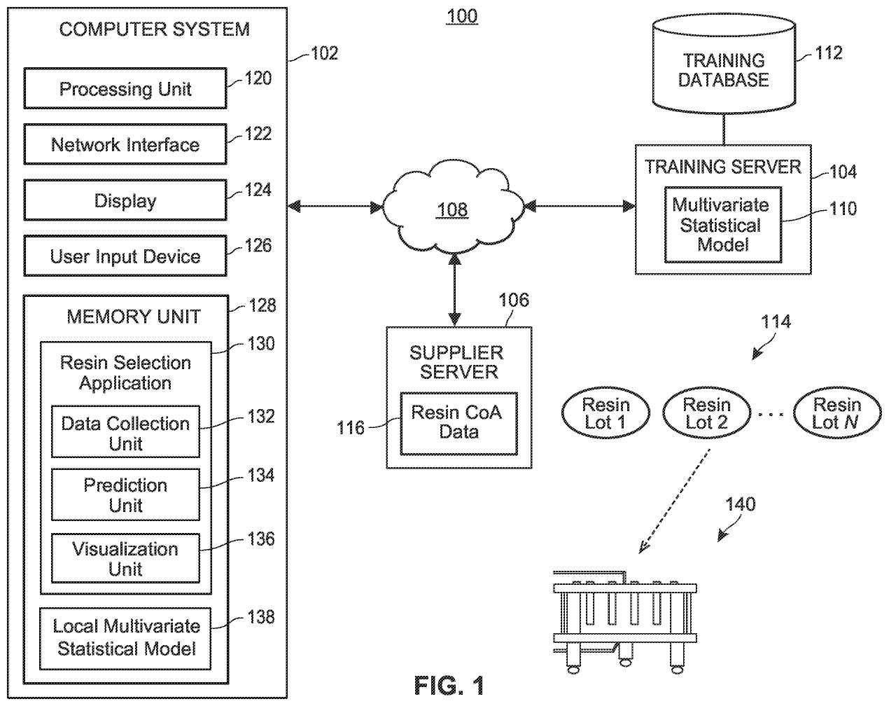

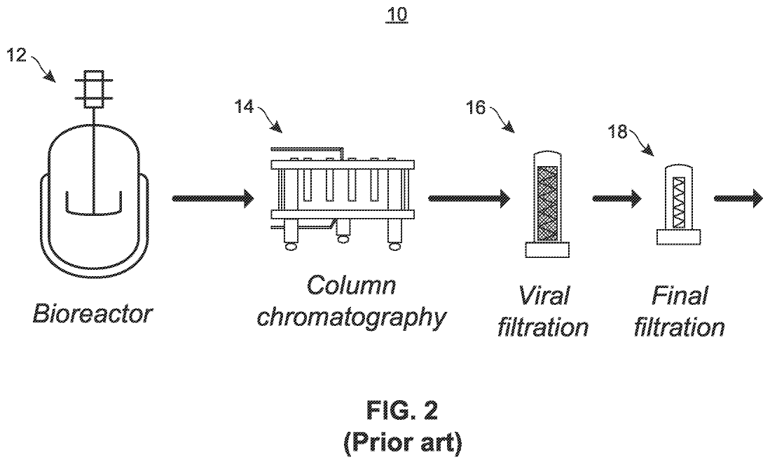

FIG. 1 is a simplified block diagram of an example system 100 that may implement the techniques described herein. System 100 includes a computing system 102 communicatively coupled to a training server 104 and a supplier server 106 via a network 108. Generally, computing system 102 and/or training server 104 are configured to train a multivariate statistical model 110 (also referred to herein as simply “model 110”) using training data in a training database 112, and use the trained model 110 to predict purification performance (e.g., a level of undesired host cell protein, or “HCP”) for a column chromatography process that may be used in the manufacture of therapeutic proteins. It should be appreciated that the “column chromatography process” may be a process that will be implemented in the future, a process that was implemented in the past, a process that is currently being implemented, or a strictly hypothetical process that never has been and never will be implemented. That is, a corresponding, real-world process may or may not exist. The column chromatography purification process may include at least one of a CEX process, an SEC process, a Protein A chromatography process, any reverse-phase chromatography process or any other suitable chromatography process.

As discussed in further detail elsewhere herein, the model 110 predicts purification performance based at least in part on attribute values for raw materials (specifically, a resin) to be used as the stationary phase in the (real or hypothetical) column chromatography purification process. In some embodiments, the model 110 is a projection on latent structures (PLS) model. As discussed in further detail elsewhere herein, a PLS model can provide a high level of accuracy when operating on resin attribute values, and possibly other input parameters, to predict a performance indicator (e.g., HCP concentration) at commercial scale. While multivariate statistical models have been proposed to predict column chromatography performance, the present embodiments can provide a substantially more reliable prediction by accounting for variability (e.g., lot-to-lot variability) in resin attribute values. In other embodiments, model 110 is another suitable type of multivariate statistical (e.g., regression) model. In some embodiments, for example, model 110 may be or include a regression (or “decision” or “ID”) tree model, an elastic net model, a lasso model, a ridge model, a support vector machine (SVM) model, etc. Moreover, in some embodiments, model 110 may include different models trained to predict different performance indicators. In some embodiments, for example, model 110 specifically includes a PLS model for predicting HCP levels, a decision tree model for predicting a level of aggregated proteins and/or protein fragments, and so on. Further, in some embodiments, model 110 may include more than one model of any given type (e.g., two or more models of the same type that are trained on different historical datasets, using different feature sets, and/or having different hyperparameters).

The attribute values operated upon by model 110 for any given run/prediction may correspond to a specific one of N resin lots 114, for example, where N is any integer greater than zero. The resin attribute values may include, for example, parameters from a certificate of analysis (“CoA”), such as any one or more from the following, non-exclusive list: pore diameter; pore volume; %20-30 um, unbounded; capacity factor; % by number 2-10 um average 3-bonded; % by volume 20-30 um average 3-bonded; mean particle size; ribonuclease retention time; insulin retention time; lysozyme retention time; myoglobin retention time; ovalbumin retention time; oxytocin retention time; bradykinin retention time; angiotensin II (angioII) retention time; neurotensin (neuro) retention time; and/or angiotensin I (angioI) retention time.

Analytical measurements of a particular one of resin lots 114 (e.g., measurements of any one or more of the types of resin attribute values noted above) may be taken by the supplier (e.g., manufacturer). Alternatively, the analytical measurements may be made by the drug manufacturer (e.g., a drug manufacturer associated with computing system 102) and/or another entity (e.g., a contractor to the resin manufacturer or drug manufacturer).

In the description that follows, attribute values for different resins may be attribute values that correspond to different resin lots (e.g., different ones of resin lots 114). It should be understood, however, that attribute values for different resins may instead correspond to different subsets of a single resin lot, to different types of resins (e.g., resins manufactured with different recipes or formulations), to resins provided by different manufacturers, and so on.

Some or all of the resin attribute values may be specified in a CoA that the manufacturer (or other supplier, etc.) provides to an entity that owns and/or maintains computing system 102 (e.g., a drug manufacturer). FIG. 1 shows an embodiment in which CoA values are stored in a memory of supplier server 106 as resin CoA data 116, which the server 106 can then electronically send to computing system 102 and/or training server 104 via network 108 (e.g., via HTTP, FTP, email, etc.). In other embodiments, however, system 100 excludes server 106, and the supplier or another entity provides resin CoA data 116 (or another form of information specifying the resin attribute values) to computing system 102 and/or training server 104 by other means, such as on papers included with the physical shipment which may include a QR code or the like to access resin CoA data from a server, or any kind of computer readable media, etc.

In addition to resin attribute values, the multivariate statistical model 110 may operate on one or more other types of inputs. For example, inputs to the model 110 may also include one or more purification process operating parameters (also referred to herein as simply “purification process parameters”), one or more harvest filtrate process performance parameters (also referred to herein as simply “harvest filtrate parameters”), and/or one or more other types of numerical and/or categorical parameters (e.g., a parameter indicating the modality of the desired therapeutic protein such as monoclonal or bispecific, etc.). Purification process parameters may include, for example, Column HETP, Column asymmetry, and/or other suitable parameters. Harvest filtrate parameters may include, for example, production bioreactors final viability, DFM individual RP-HPLC total area, DFM individual RP-HPLC main area, DFM individual RP-HPLC impurity area, DFM Individual PI, DFM individual PI titer, and/or other suitable parameters, where DFM=diafiltered medium, HETP=height equivalent of a theoretical plate, PI=product isoform, and RP-HPLC=reverse phase high performance liquid chromatography.

Computing system 102 may also be generally configured to enable one or more users, who may be local or remotely distributed, to make use of the prediction capabilities of computing system 102, and to provide various interactive capabilities to the user(s) as discussed elsewhere herein.

Network 108 may be a single communication network, or may include multiple communication networks of one or more types (e.g., one or more wired and/or wireless local area networks (LANs), and/or one or more wired and/or wireless wide area networks (WANs) such as the Internet). In various embodiments, training server 104 may train and/or utilize the multivariate statistical model 110 as a “cloud” service (e.g., Amazon Web Services), or training server 104 may be a local server. In the depicted embodiment, however, model 110 is trained by server 104, and then transferred to computing system 102 via network 108 as needed. In other embodiments, model 110 is trained on computing system 102, and then uploaded to training server 104 for later access. In still other embodiments, computing system 102 trains and maintains/stores the multivariate statistical model 110, in which case system 100 may omit training server 104 (and possibly network 108), or server 104 may be a part of computing system 102.

Computing system 102 may include one or more general-purpose computers specifically programmed to perform the operations discussed herein, and/or may include one or more special-purpose computing devices. As seen in FIG. 1, computing system 102 includes a processing unit 120, a network interface 122, a display 124, a user input device 126, and a memory unit 128. In embodiments where computing system 102 includes two or more computers (either co-located or remote from each other), the operations described herein relating to at least processing unit 120, network interface 122, and/or memory unit 128 may be divided among multiple processing units, multiple network interfaces, and/or multiple memory units, respectively. Moreover, display 124 and user input device 126, while referred to herein in the singular, may include multiple displays and multiple user input devices, respectively. For example, display 124 may include at least one display at each of a number of remote, user-specific client devices, and user input device 126 may include at least one user input device for each of those client devices.

Processing unit 120 includes one or more processors, each of which may be a programmable microprocessor that executes software instructions stored in memory unit 128 to execute some or all of the functions of computing system 102 as described herein. Processing unit 120 may include one or more central processing units (CPUs) and/or one or more graphics processing units (GPUs), for example. Alternatively, or in addition, some of the processors in processing unit 120 may be other types of processors (e.g., application-specific integrated circuits (ASICs), field-programmable gate arrays (FPGAs), etc.), and some of the functionality of computing system 102 as described herein may instead be implemented in hardware.

Network interface 122 may include any suitable hardware (e.g., a front-end transmitter and receiver hardware), firmware, and/or software configured to communicate with training server 104 via network 108 using one or more wired and/or wireless communication protocols. For example, network interface 122 may be or include a WiFi or Ethernet interface, enabling computing system 102 to communicate with training server 104 over the Internet or an intranet, etc.

Display 124 may use any suitable display technology (e.g., LED, OLED, LCD, etc.) to present information to a user, and user input device 126 may be a keyboard or other suitable input device. In some embodiments, display 124 and user input device 126 are integrated within a single device (e.g., a touchscreen display). Generally, display 124 and user input device 126 may combine to enable a user to interact with graphical user interfaces (GUIs) provided by computing system 102. However, computing system 102 may omit display 124 and/or user input device 126, e.g., in certain embodiments where computing system 102 interacts with other computing devices or systems (e.g., client devices of third parties) to enable interaction by users of those devices or systems.

Memory unit 128 may include one or more volatile and/or non-volatile memories. Any suitable memory type or types may be included, such as read-only memory (ROM), random access memory (RAM), flash memory, a solid-state drive (SSD), a hard disk drive (HDD), and so on. Collectively, memory unit 128 may store one or more software applications, the data received/used by those applications, and the data output/generated by those applications. These applications include a resin selection application 130 that, when executed by processing unit 120, predicts and presents performance of a virtual (in silico) column chromatography process for purification during therapeutic protein manufacture. In some embodiments, the various “units” of resin selection application 130 discussed herein may be distributed among different software applications, and/or the functionality of any one such unit may be divided among two or more software applications.

In the example system 100, resin selection application 130 includes a data collection unit 132, a prediction unit 134, and a visualization unit 136. In general, data collection unit 132 receives (e.g., retrieves) the parameters that prediction unit 134 applies as inputs to a local multivariate statistical model 138, to predict the performance indicator. In the depicted embodiment, model 138 is a local copy of the model 110 trained by training server 104, and may be stored in a RAM or ROM of memory unit 128, for example. As noted above, however, training server 104 may utilize/run multivariate statistical model 110 in some embodiments, in which case no local copy need be present in memory unit 128, or multivariate statistical model 110 may originally reside in a persistent memory of memory unit 128 rather than being retrieved from training server 104 on an as-needed basis. Data collection unit 132 may receive the resin attribute values (e.g., resin CoA data 116) from supplier server 106 via network 108, and may receive other parameters operated upon by local multivariate statistical model 138 from a user entering parameters/values via a GUI (e.g., presented on display 124) that is generated or populated by visualization unit 136, and/or as one or more files or other data transfers (e.g., using file paths designated by a user via such a GUI), for example.

Visualization unit 136 may also generate and/or populate a GUI to view and/or interact with the predicted results of the modeled process (e.g., values of the performance indicator output by prediction unit 134 using model local multivariate statistical model 138), for example. For example, visualization unit 136 may cause the GUI to display the predicted HCP concentration (or concentration of another impurity type, or a total impurity concentration, etc.) for a given set of resin attribute values that correspond to a particular one of resin lots 114, as well as any other parameters used as inputs to local multivariate statistical model 138 (e.g., values of various process parameters and/or harvest filtrate parameters).

Operation of system 100, according to one embodiment, will now be described in further detail. Initially, training server 104 trains multivariate statistical model 110 using historical data stored in a training database 112. Training database 112 may include a single database stored in a single memory (e.g., HDD, SSD, etc.), or may include multiple databases stored in one or more memories. In some embodiments, and as discussed in further detail herein, various techniques (e.g., small-scale modeling) may be used to identify which features (e.g., resin attribute values, purification process parameters, etc.) are most predictive of a particular performance indicator, and/or may be trained or re-trained using a feature set that only includes the features that are most predictive of a particular performance indicator. While multivariate statistical model 110 may include multiple, distinct models, for ease of explanation the description herein refers to multivariate statistical model 110 in the singular, and it is understood that the techniques described herein can be applied to multiple models.

Training database 112 stores a set of training data to train multivariate statistical model 110 (e.g., input/feature data, and corresponding labels). To train a model that predicts HCP concentration, for instance, training database 112 may include numerous sets of inputs/features each comprising historical resin attribute values (and possibly purification process parameters and/or harvest filtrate parameters, etc.), along with a known (e.g., measured) HCP concentration corresponding to each feature set. In some embodiments, all features and labels are numerical, with non-numerical classifications or categories being mapped to numerical values (e.g., with the allowable values [Monoclonal, Bispecific Format 1, Bispecific Format 2, Bispecific Format 1 or 2] of a modality feature/input being mapped to the values [00, 10, 01, 11]).

In some embodiments, training server 104 uses additional labeled data sets in training database 112 in order to confirm/validate the trained multivariate statistical model 110 (e.g., to confirm that multivariate statistical model 110 provides at least some minimum acceptable accuracy). In some embodiments, training server 104 also updates/refines multivariate statistical model 110 on an ongoing basis. For example, after multivariate statistical model 110 is initially trained to provide a sufficient level of accuracy, and is put into use for a commercial-scale process (e.g., to screen resin lots), additional measurements of the performance indicator (and corresponding input/features) at commercial scale may be used to further improve prediction accuracy of the multivariate statistical model 110.

Resin selection application 130 may at some later point then retrieve, from training server 104 via network 108 and network interface 122, a copy of multivariate statistical model 110. Upon retrieving the model, computing system 102 stores a local copy as local multivariate statistical model 138. In other embodiments, as noted above, no model is retrieved, and input/feature data is instead sent to training server 104 (or another server) as needed to use the multivariate statistical model 110, or multivariate statistical model 110 may reside only at computing system 102.

In accordance with the feature set for which local multivariate statistical model 138 is designed/trained, data collection unit 132 collects the necessary data. For example, data collection unit 132 may receive resin CoA data 116 from supplier server 106 or via user entry of information (e.g., on a GUI presented on display 124). Data collection unit 132 also collects any other parameters used as model inputs, such as user-entered purification process parameters and/or harvest filtrate parameters, for example. After data collection unit 132 has collected the model inputs for a particular candidate resin (e.g., one of resin lots 114), prediction unit 134 causes local multivariate statistical model 138 to operate on those inputs/features to predict the desired performance indicator for the column chromatography process. In some embodiments, the local multivariate statistical model 138 predicts HCP levels (e.g., HCP concentration), and the data collection unit 132 collects resin attribute values (e.g., values of any one or more of the example CoA parameters listed above), purification process parameter values (e.g., values of any one or more of the example purification process parameters listed above), and harvest filtrate parameters (e.g., values of any one or more of the example harvest filtrate parameters listed above) for use as inputs to local multivariate statistical model 138.

Visualization unit 136 may then cause a GUI, depicted on display 124, to present the predicted performance indicator, and/or other information based on the predicted performance indicator (e.g., a list/ranking of predicted performance indicators for different resin lots, a binary indication of whether the predicted performance indicator is “acceptable” as compared to a predetermined threshold, etc.). Visualization unit 136 may also cause the GUI to present confidence metrics associated with predicted performance indicators (e.g., confidence metrics generated by local multivariate statistical model 138). For example, the GUI may display a range of HCP levels that correspond to at least a 90% confidence level (or 80%, 95%, etc.). If repeated for multiple resin lots 114, a user can then select which resin lots (or resin types, etc.) are acceptable or unacceptable (e.g., by comparing the different predictions to each other, or by comparing each prediction to an acceptability threshold, etc.). In general, for any given one of resin lots 114, the user may use the displayed prediction and/or result (possibly in conjunction with other information) to determine whether the lot is acceptable. If the lot is acceptable, the user may select that lot (e.g., indicate approval of the lot via the GUI or other means) for use in a real-world column chromatography process. To this end, the example system 100 includes a real-world column chromatography system 140 that is configured to perform a column chromatography process (e.g., a CEX, SEC, Protein A, or other type of column chromatography), and the selected/accepted resin may be used as the stationary phase for that process. The column chromatography system 140 may be a commercial-scale column chromatography system used during the commercial manufacture of a therapeutic protein, for example. Depending on the embodiments, the column chromatography system 140 may include one or more columns, and the selected resin may be used for one, some, or all of those columns.

The prediction/visualization process may be performed just once (e.g., when screening a single received resin lot to determine whether the lot is usable), or multiple times (e.g., when selecting which of multiple received resin lots 114 are to be kept, or ordered in the first instance, etc.). Whether the goal is to screen a single resin lot or select from among multiple candidate resin lots, this process of predicting and visualizing purification performance can substantially reduce the amount of drug substance that must be rejected/discarded due to poor purification performance, and/or substantially reduce the amount of time needed to ensure acceptable purification performance. For example, the time required to screen (i.e., ensure acceptable performance for) one or more resin lots may be reduced from tens or even hundreds of hours down to something on the order of one or two hours.

In other embodiments or scenarios, the process may be repeated as many times as desired for purely hypothetical resin attribute values, e.g., in order to identify critical resin attributes, identify optimal resin attribute values or value ranges, or identify ways in which different resin attributes interact with other parameters (e.g., to assess correlations of harvest filtrate and/or chromatography process parameters with specific resin attributes), and so on. Results from these virtual experiments can be conveyed to manufacturers as needed (e.g., to enable the manufacturer to vary its resin formulation/recipe accordingly).

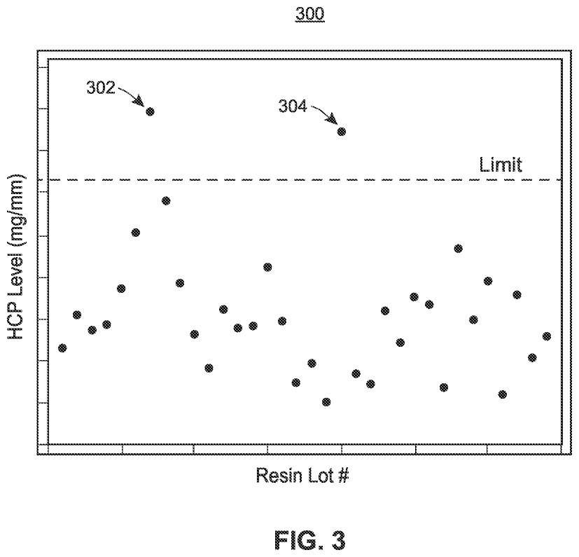

Various techniques may also be used, separately or in conjunction with (e.g., as a precursor to) the statistical modeling techniques described herein, to gain better insight into how variability in resin attributes can affect the column chromatography purification process. For example, while conventional risk-based analyses may ensure that most resin lots result in acceptable purification performance, unexpected outliers may nonetheless occur. FIG. 3 depicts an example of two such instances. In chart 300 of FIG. 3, the measured lot-to-lot variability in commercial-scale purification performance (specifically, HCP level/concentration) is shown across a number of different resin lots over a particular time period. As can be seen in the chart 300, most resin lots resulted in a seemingly safe degree of clearance relative to the acceptability limit/threshold for the resulting HCP level. However, changes in attributes of the manufactured resin over time resulted in the acceptability limit being neared and, eventually, exceeded at both a first event 302 and a later second event 304.

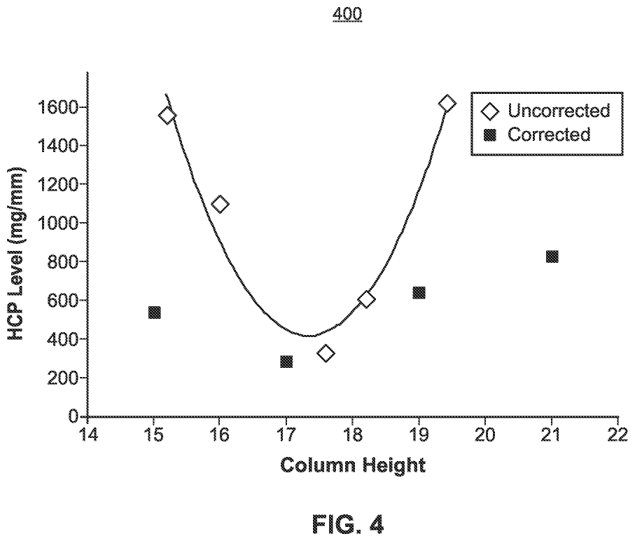

Construction and implementation of a small-scale column chromatography model, designed to replicate in important respects (but in smaller dimensions/amounts) the commercial-scale column chromatography system that resulted in the HCP levels shown in FIG. 3, led to a successful re-creation of the deviation shown as the first event 302 of FIG. 3. By varying the height of the resin column (also referred to as the “bed height” or “packed bed height”) using this small-scale model, it was determined that column height variability impacts chromatographic performance, with a generally quadratic response as seen in chart 400 of FIG. 4. In FIG. 4, process parameters for the small-scale model were established based on the assumed column height, and then adjusted based on the actual/observed column height. The root cause of the first event 302 was attributed to the column height and its effect on the operating parameters of flow rate, wash volume, and elution gradient slope. Corrective actions were then taken based on these learned relationships, including (1) narrowing the normal operating range (NOR) of the process to decrease variability in the bed height, and (2) establishing automation set points for flow rate, wash volume, and gradient slope based on the actual measured bed height (and not on a theoretical value).

The small-scale model was also used to characterize various other aspects of the commercial-scale column chromatography process, in order to optimize operational parameters (e.g., gradient, temperature, buffer concentrations, gradient start, gradient end, etc.) without impacting other product attributes. Moreover, tighter ranges for operational parameters were identified by taking into consideration equipment control tolerances and risk simulations.

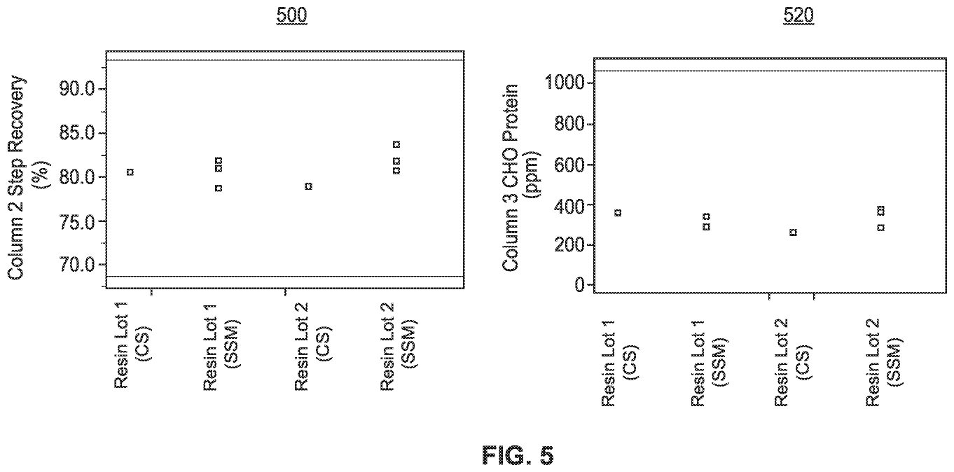

FIG. 5 includes charts 500 and 520 comparing small-scale model and commercial-scale purification performance with respect to step recovery percentage (in the second column of a four-column purification system) and HCP level (in the third column of a four-column purification system), respectively. As can be seen in FIG. 5, the small-scale model (SSM) performance closely approximates the commercial-scale (CS) performance. Accordingly, the small-scale model can be used for resin screening prior to commercial-scale use. In this manner, good performance for purification at commercial scale can be maintained despite lot-to-lot resin variability (e.g., poor performing resin lots can be discarded or returned, or used in conjunction with additional purification steps, etc.). However, screening via a small-scale model of a column chromatography system (e.g., a small-scale model of column chromatography system 140 of FIG. 1) can require tens or even hundreds of hours, as compared to something potentially on the order of one or two hours using the multivariate statistical model described herein (e.g., multivariate statistical model 110 or local multivariate statistical 138 of FIG. 1). Thus, it may be preferable to use small-scale modeling to gain certain insights about resin attributes and their interactions with various chromatography operational parameters, but instead use a multivariate statistical model to screen resin lots.

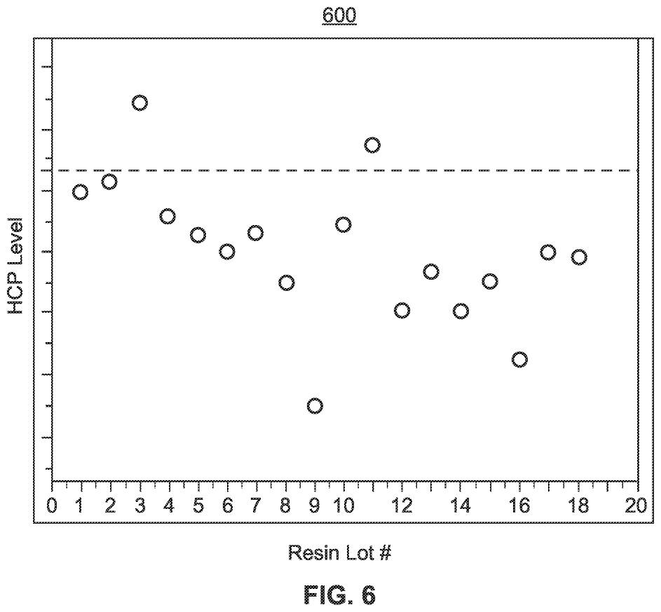

FIG. 6 depicts a chart 600 showing experimental purification performance (HCP levels) for different resin lots using a small-scale model of a commercial-scale column chromatography system. As can be seen, this small-scale model identified two out of 18 resin lots as being unsatisfactory (i.e., exceeding the acceptable HCP threshold/limit). Thus, the two underperforming resin lots could be screened out, rather than using the lots at commercial scale only to arrive at unacceptable purity levels.

Referring again to FIG. 3, several corrective modifications were implemented for the commercial-scale chromatography system after the first event 302, based on the results from the small-scale model. For example, the above-noted adjustments relating to bed height, flow rate, wash volume, and gradient slope were implemented at commercial scale. Nonetheless, another event at commercial scale was observed years later (i.e., the second event 304 of FIG. 3), with the resulting HCP level again exceeding the acceptable limit, despite the small-scale model having identified the resin lot as acceptable.

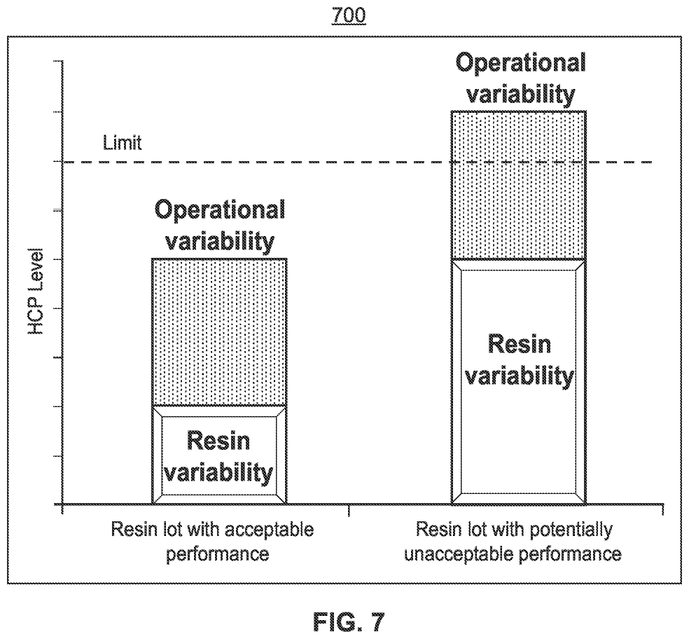

An investigation of the second event 304 revealed that, in addition to resin attributes for a particular lot, the process harvest filtrate had a significant influence on resulting HCP levels. This was supported by characterization data showing the variability in HCP levels between different lots of harvest filtrate. FIG. 7 depicts a chart 700 showing the combined/total variability in HCP levels resulting from both resin lot-to-lot variability and operational variability, where “operational variability” refers to the combination of (1) the variability of purification process parameters that were identified during the first event 302 and (2) the lot-to-lot variability of the harvest filtrate. Purification process parameters that may vary can include, for example, Column 1 HETP, Column 1 asymmetry, and/or other parameters. Harvest filtrate parameters that may vary can include, for example, production bioreactor final viability, DFM individual RP-HPLC total area, DFM individual RP-HPLC main area, DFM individual RP-HPLC impurity area, DFM Individual PI, DFM individual PI titer, and/or other parameters. As can be seen in chart 700, when viewed in isolation (i.e., without operational variability), resin lot-to-lot variability can appear to indicate acceptable HCP clearance relative to the acceptability threshold even in instances where, in fact, the threshold might be exceeded.

To better understand how resin lot variability contributes to HCP variability, a collaboration study with a resin manufacturer was undertaken. The collaboration study focused on understanding which resin manufacturing parameters/attributes significantly influenced HCP clearance at the chromatography stage, in order to control those attributes and ensure purification performance consistency. In the collaboration study, the chromatography loading material (representative of the material used at commercial scale) was used for small-scale modeling, and purification was performed with 1.0 cm inner diameter Omnifit® Benchmark chromatography columns. Small-scale model runs were performed using GE® Healthcare ÄKTA Explorer® 100 systems with UNICORN® 5.0 software. All solutions and buffers used were prepared using raw materials from qualified suppliers, and following small-scale recipes. HCP levels/results were evaluated using an enzyme-linked immunosorbent assay (ELISA). The experimental design construction and model analysis for the experiments were performed using JMP® statistical discovery software from SAS®.

In the collaboration study, a full factorial design-of-experiments (DOE) method, including center point runs to test the lack of fit due to nonlinear effects, was performed for three resin attributes: ligand A level, ligand B level, and end-capper level. The selection of these attributes was based on a scientific rationale relating to the known protein binding mechanism and mixed mode interaction. For the study, the three attributes were modified within a normal operating range (NOR), resulting in nine different permutations that were used by the manufacturer to generate different resin samples. Once manufactured, the resin samples were used as the stationary phase for the small-scale model. The HCP results were then used to evaluate the small-scale model based on an adjusted actual-by-predicted plot of the coefficient of determination (“R² adj”). Thereafter, the small-scale model was used to identify the main effects, factors, and interactions that contributed to the HCP results.

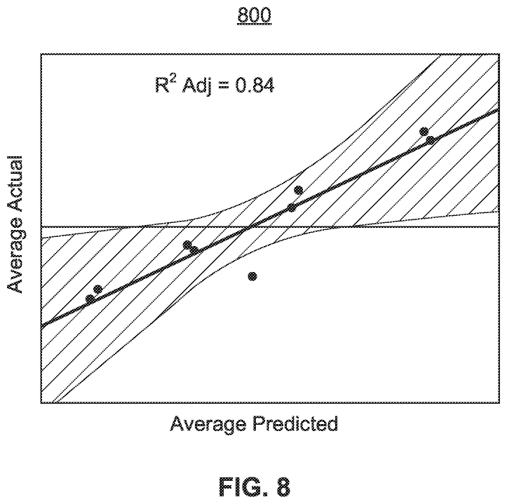

From the DOE results, a Fit Model stepwise regression analysis was performed to evaluate the effect of one factor, and to model multi-factor interactions, on the response variable (i.e., HCP level). As shown in chart 800 of FIG. 8, the regression analysis resulted in an actual-by-predicted plot with an adjusted coefficient of determination (R² Adj) of 0.84, which confirms the high level of predictability offered by the model. The R² Adj statistic is a modified version of the coefficient of determination (R²), and compares the descriptive power of regression models that include a diverse number of predictors. Every predictor added to the model increases the R² for that model. Thus, whereas a model with more terms may seem to have a better fit simply because it has more terms, R² Adj compensates for the addition of variables. R² Adj only increases if a new term enhances the model beyond what could result by chance, and only decreases if a new term degrades the model beyond what could result by chance.

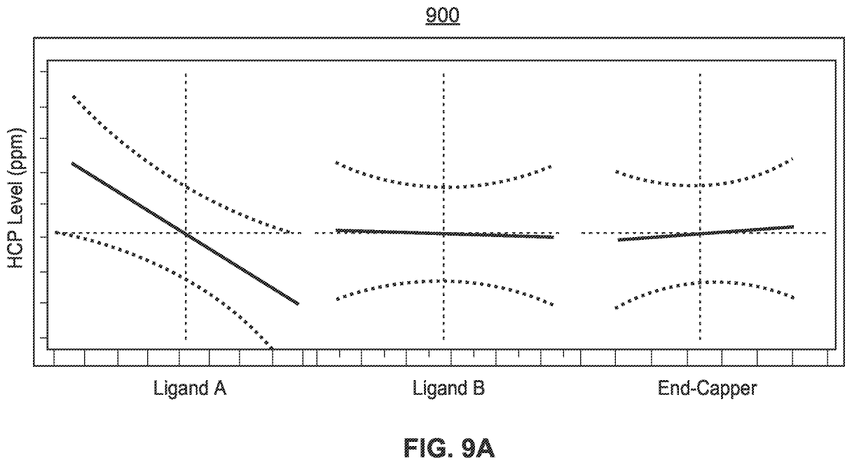

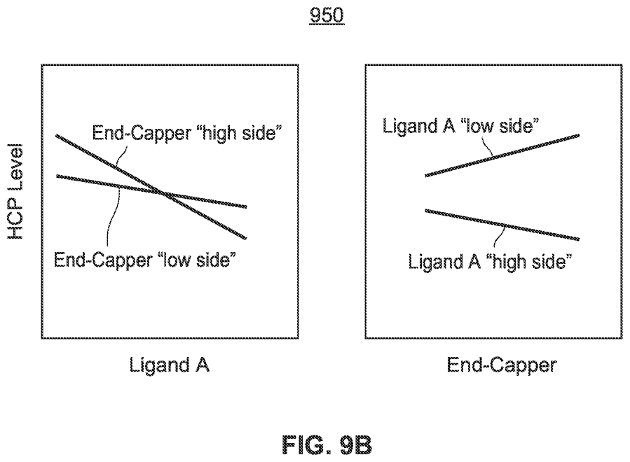

Of the three factors evaluated (ligand A, ligand B, and end-capper levels), a statistically significant effect on HCP level/clearance (here defined as the p-value being less than 0.05) was only obtained for the ligand A level (p-value 0.0020), and for the interaction between ligand A and end-capper levels (p-value 0.0277), with the ligand A level being by far the most significant. The relation between HCP level and each of ligand A, ligand B, and end-capper level is shown in the charts 900 of FIG. 9A. In the charts 900, a larger slope corresponds to a greater effect on HCP levels/clearance. FIG. 9B depicts charts 950 showing the interaction between ligand A level, end-capper level, and HCP level/clearance. As seen in the charts 950, the best performance (lowest HCP level) results from the combination of a relatively high ligand A level and a relatively high end-capper level.

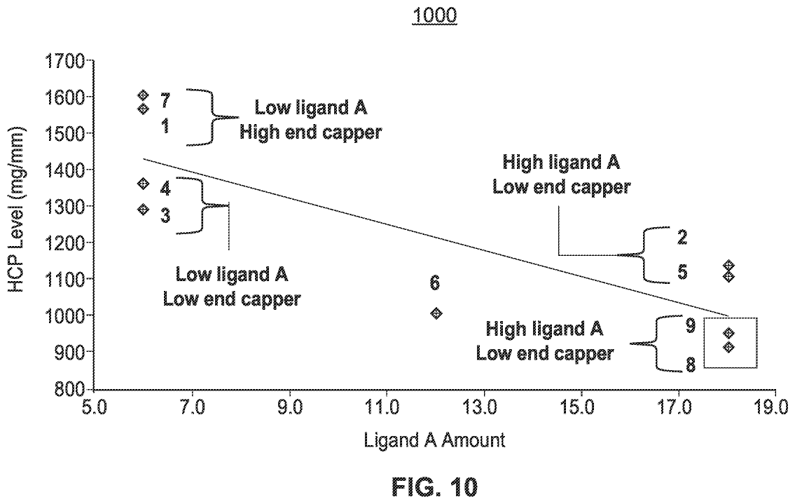

FIG. 10 depicts a chart 1000 providing more detailed results. Chart 1000 shows HCP results for nine test runs labeled “1” through “9,” with each run corresponding to a different permutation of relatively high levels (“+”), relatively low levels (“−”), or intermediate levels (“0”) for the three resin attributes as shown below in Table 1:

TABLE 1

| RUN | Ligand A Level | Ligand B Level | End-Capper Level |
|---|---|---|---|
| 1 | − | − | + |
| 2 | + | + | − |
| 3 | − | + | − |
| 4 | − | − | − |
| 5 | + | − | − |
| 6 | 0 | 0 | 0 |
| 7 | − | + | + |
| 8 | + | + | + |
| 9 | + | − | + |

As seen in chart 1000, run numbers 8 and 9 provide the best HCP results from among the nine runs, with ligand B levels showing only a very slight effect on performance.

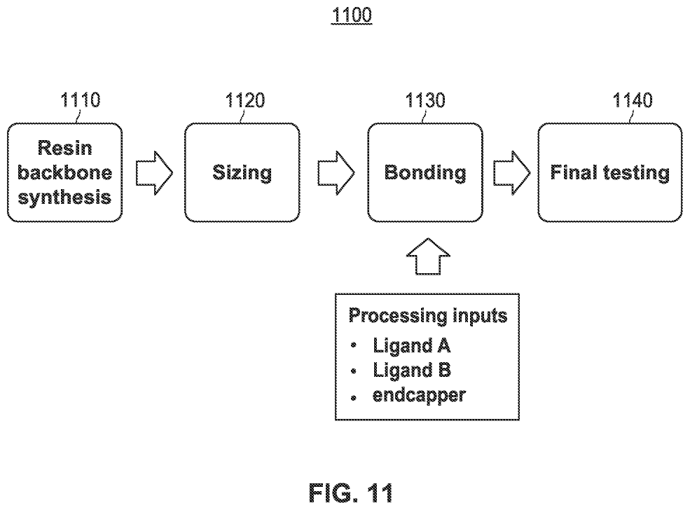

A resin manufacturer can use this information to improve the resin manufacturing process. An example of one such process 1100 is shown in FIG. 11. In the process 1100, resin manufacturing begins with synthesis 1110 of the resin backbone, followed by sizing 1120 and bonding 1130 steps, and then final testing 1140. Based on the resin attribute values determined from the collaboration study, changes may be made at the bonding 1130 step. While FIG. 11 shows inputs relating to ligand A, ligand B, and end-capper levels, it may be sufficient to adjust the resin manufacturing process 1100 based only on desired ligand A levels (given the relatively small effects of end-capper and ligand B levels), or based only the desired combination of ligand A and end-capper levels (given the particularly small effect of ligand B levels).

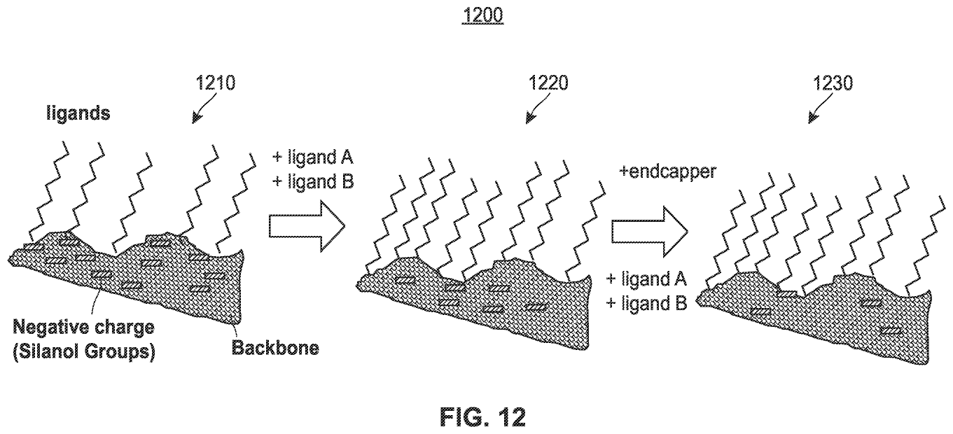

FIG. 12 depicts example modifications 1200 to the resin manufacturing process (e.g., process 1100), based on the small-scale model results (shown in FIGS. 9 and 10, and in Table 1) that improved purification performance. As seen in FIG. 12, a current state 1210 represents a resin manufacturing process prior to the input/feedback provided to the resin manufacturer at step 1130 of FIG. 11. The resin backbone, which includes negatively charged silanol groups, is bonded to ligands including ligand A and ligand B. A first modification stage 1220 involves increasing the concentration/amount of both ligand A and ligand B, to increase the carbon load and surface coverage on the backbone. Optionally, ligand B may not be increased, due to its relatively small effect on performance. A second modification stage 1230 involves further increasing the ligand A and ligand B concentrations, and also increasing the end-capper level, to increase the carbon load and surface coverage on the backbone, and to decrease the available silanol groups to affect mixed mode interaction.

The data obtained from the collaboration study DOE results, from analysis of the harvest filtrate contribution, and from historical commercial-scale and small-scale model historical HCP levels/results was used to generate a multivariate statistical predictive model, with the goal of providing a more efficient and robust resin screening process, prior to using resins in a commercial-scale column chromatography purification process. The resulting model may be used, for example, as multivariate statistical model 110 and/or local multivariate statistical model 138 of FIG. 1. In this case, the multivariate statistical model was a projection on latent structures (PLS) model, with the goal being to establish a correlation between process parameters and process responses, and to identify the parameters that most influence the quality of process responses. While a PLS model was built and tested in this case, it is understood that other embodiments may instead use other types of models (e.g., elastic net, decision tree, etc.).

The data used to train the PLS model was a collection from cell culture harvest filtrate, purification process parameters, and resin attribute values (i.e., resin attribute values as specified on CoAs for various resin lots). The data reflected a number of “observations,” which were divided into training and validation/confirmation subsets. The confirmation data set included drug substance batches randomly selected across a span of commercial-scale HCP results/levels. The training data set included the remainder of the drug substance batches, as well as data from small-scale model runs. In any given drug substance batch or small-scale model run, a different blend of harvest filtrate loading material and/or resin lots may have been used. Thus, each training input (or “x-variable”) was expanded to three inputs/x-variables (e.g., minimum, maximum, and weighted average), in order to better capture potential contributions to the PLS model and prediction of the output (“y-variable,” here HCP level) at the chromatography step under evaluation.

The training and confirmation data sets were then processed to generate and validate a first iteration of the PLS model. This was performed using SIMCA 14.1 tools from Umetrics®, although newer versions may be used when updating the model with more recent data (i.e., to expand the training set and thus the predictability range). The predictive power of each input/x-variable was then determined and analyzed. To this end, the SIMCA 14.1 tools were used to generate a plot showing the variable importance for the projection (or “VIP”) of each x-variable. X-variables with higher VIP values have a greater contribution to the fit and predictability of the model. From among the minimum/maximum/weighted average values associated with each x-variable, only the one with the highest VIP value was retained/used for the next iteration of the PLS model.

Once the final trained PLS model was created, the outputs/y-variables (predicted HCP levels) for the confirmation set were predicted and compared against the actual/known values (measured HCP levels). The fitness and predictability of the final PLS model was assessed based on various types of information, such as a residuals plot (i.e., a plot of residuals standardized on a double log scale), a permutations plot (i.e., a plot reflecting variations in the portions of the data set used for training and for confirmation, to assess the risk that the current PLS model fits the training data set well but does not predict the output well for new observations), a VIP plot (i.e., to summarize the importance of the variables, both for explaining inputs/x-variables and correlating to the output/y-variable), and a plot that displays the observed values versus the predicted values of the output/y-variable.

Six iterations of the PLS model (each with two principal components) were generated, with the following performance metrics:

TABLE 2

| Model | R²X | R²Y | Q² |
|---|---|---|---|
| M1 | 0.472 | 0.862 | 0.831 |
| M2 | 0.523 | 0.843 | 0.815 |
| M3 | 0.517 | 0.850 | 0.820 |
| M4 | 0.532 | 0.851 | 0.822 |
| M5 | 0.549 | 0.850 | 0.824 |
| M6 | 0.570 | 0.848 | 0.822 |

R² is a measure of how well the model fits the data set (with R²X measuring the fit in inputs and R²Y measuring the fit in output/HCP), and Q² is a measure of how predictive/accurate the model is. The goal is to maximize R²Y, although other factors may also be considered, such as simplicity of the model (e.g., number of inputs).

The model resulting from the final iteration (M6) had an R²Y value (0.848) slightly below the highest R²Y value (0.862), but had the advantage of being trained on fewer x-variables than other models. Specifically, M6 was trained on an input set consisting of 17 resin attribute values from a CoA (pore diameter, pore volume, %20-30 um unbounded, capacity factor, % by number 2-10 um average 3-bonded, % by volume 20-30 um average 3-bonded, mean particle size, ribonuclease retention time, insulin retention time, lysozyme retention time, myoglobin retention time, ovalbumin retention time, oxytocin retention time, bradykinin retention time, angioII retention time, neuro retention time, and angioI retention time), six harvest filtrate loading material parameters (production bioreactor final viability, DFM individual RP-HPLC total area, DFM individual RP-HPLC main area, DFM individual RP-HPLC impurity area, DFM Individual PI, DFM individual PI titer), and two downstream purification process parameters (Column 1 HETP, Column 1 asymmetry). A normal probability plot of residuals showed no outliers in the final (M6) PLS model (with all probabilities falling within plus or minus four standard deviations). Moreover, a permutation plot showed that the final PLS model was a unique solution to the training data set. More specifically, a plot of R²Y and Q² values versus the correlation between the permuted y-variable and the original y-variable showed a large, clear separation between values for the original M6 model and values for all permutations of the M6 model (i.e., with the original M6 values of R²Y and Q² being 0.848 and 0.822, respectively, and the permutation values all being less than about 0.3 or less than about 0.1, respectively). The permutation plot also showed that the regression line for Q² fell below zero, which further indicates that the PLS model was a unique solution to the data set.

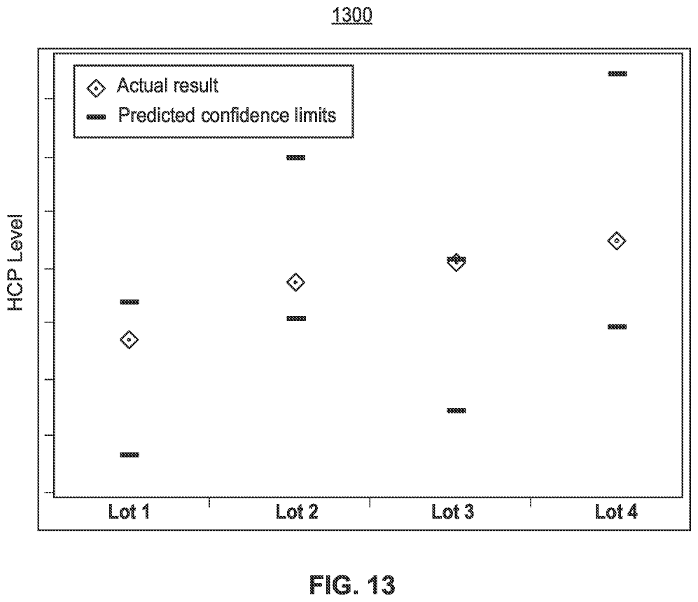

FIG. 13 depicts a chart 1300 that compares actual purification performance (in this example, HCP levels) with confidence limits predicted by the M6 model, for four different drug substance lots not 1″ through “Lot 4”) represented in the confirmation data set. As seen in chart 1300, in all of the drug substance lots in the confirmation data set, the actual HCP levels fell within the predicted HCP range. In addition, the absolute differences between the actual and predicted values fell within the root mean square error from cross-validation, or “RMSECV.” The M6 model demonstrated accuracy similar to the small-scale model screening described herein, but required less resource usage (e.g., less labor), no analytical testing, and a vast reduction in execution and result turnaround times (i.e., from 80 hours to two hours for execution times, and from 672 hours to two hours for result turnaround times, approximately). Another advantage of a predictive (e.g., PLS) multivariate statistical model over small-scale model screening is that the former enables a user to evaluate any combination of harvest loading material and resin attribute values in silico. As with the small-scale model HCP predictions, the multivariate statistical model predictions assume that the downstream portion of the drug substance manufacturing process is executed within a normal operating range (NOR).

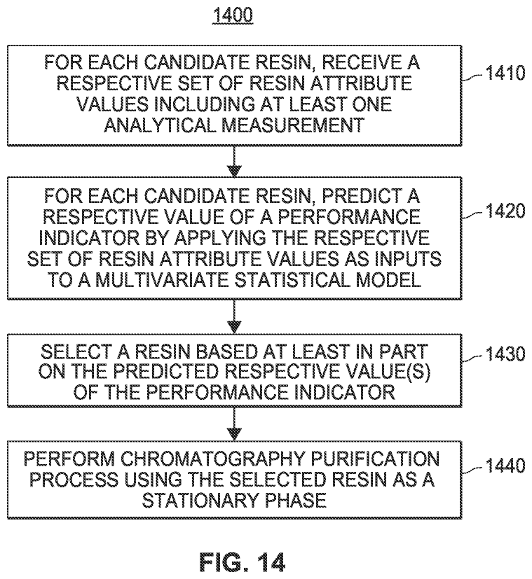

FIG. 14 is a flow diagram depicting an example method 1400 of selecting raw materials for use in a chromatography purification process, e.g., for the manufacture of therapeutic proteins. The method 1400 may be implemented, in part (e.g., blocks 1410 through either 1420 or 1430), by processing unit 120 of computing system 102 (when executing the software instructions of resin selection application 130 stored in memory unit 128), or by one or more processors of training server 104 (e.g., in a cloud service implementation), for example.

At block 1410, for each of one or more candidate resins, a respective set of resin attribute values is received, with each set including at least one analytical measurement of the candidate resin. If the method 1400 is used to screen individual resin lots, for example, block 1410 may include receiving a single set of resin attribute values for a single resin lot. As another example, if the method 1400 is used to select from among different resin products or different resin lots offered by a manufacturer, block 1410 may include receiving multiple sets of resin attribute values corresponding to the different resin products or lots. Some or all of the resin attribute values may be received (directly or indirectly) from a manufacturer or supplier of the candidate resin(s), e.g., in a CoA or other format. For example, the resin manufacturer or supplier may make any analytical measurement(s) required to obtain the CoA data, and then physically or electronically provide the CoA to an entity (e.g., drug manufacturer) that is performing the method 1400. As examples, the resin attribute values may include one or more of pore diameter, pore volume, %20-30 um unbounded, capacity factor, % by number 2-10 um average 3-bonded, % by volume 20-30 um average 3-bonded, mean particle size, ribonuclease retention time, insulin retention time, lysozyme retention time, myoglobin retention time, ovalbumin retention time, oxytocin retention time, bradykinin retention time, angioII retention time, neuro retention time, and/or angioI retention time. In some embodiments, block 1410 includes receiving data that is manually entered by a user (e.g., a user entering data from a CoA).

At block 1420, for each of the one or more candidate resins, a respective value of a performance indicator (for the column chromatography purification process) is predicted by applying the respective set of resin attribute values as inputs to a multivariate statistical model (e.g., multivariate statistical model 110 or local multivariate statistical model 138 of FIG. 1). It should be understood that the “applying” and “predicting” of block 1420 may include directly operating the model (e.g., as a local copy such as local multivariate statistical model 138), or may include triggering the operation of the model (e.g., by sending input values to training server 104 via network 108 and requesting a prediction/result, etc.).

The model may be a projection on latent spaces (PLS) model, for example, with any suitable number of principal components (e.g., two principal components). Alternatively, the model may be any other suitable type of multivariate statistical model (e.g., elastic net, decision tree, etc.). The performance indicator may be an HCP level (e.g., concentration) resulting from the column chromatography purification process, for example. Alternatively, the performance indicator may be the level of another type of impurity (e.g., aggregated proteins, protein fragments, etc.), a total level of all impurity types, or any other suitable indicator of the purity of the material that results from the column chromatography purification process. In some embodiments, each respective “value” is a range of values. For example, the model may output a range of values for which some minimum confidence level (e.g., 90%, 95%, etc.) is exceeded.

In some embodiments, block 1420 also includes applying one or more other types of inputs to the multivariate statistical model, along with the resin attribute values. For example, block 1420 may include applying one or more harvest filtrate parameter values (e.g., production bioreactor final viability, DFM individual RP-HPLC total area, DFM individual RP-HPLC main area, DFM individual RP-HPLC impurity area, DFM Individual PI, DFM individual PI titer, and/or other parameters), and/or one or more chromatography/purification process parameter values (e.g., Column 1 HETP, Column 1 asymmetry, and/or other parameters) as inputs to the multivariate statistical model.

At block 1430, a resin, of the one or more candidate resins, is selected, based at least in part on the predicted respective value(s) of the performance indicator. The “selection” may be the confirmation or approval of a particular resin lot, for example, or a designation of a particular resin type or lot as being acceptable, etc. In some embodiments, block 1430 is performed automatically by software (e.g., by processing unit 120 of computing system 102 when executing the software instructions of resin selection application 130, or by one or more processors of training server 104, etc.). Alternatively, block 1430 may be wholly or partially performed by one or more users, by considering the predicted value(s). To this end, block 1430 may include causing a user interface (e.g., a GUI generated or populated by visualization unit 136 and presented on display 124 of FIG. 1) to present the predicted performance indicator value(s), and/or a result based on the predicted value(s) (e.g., an indication of whether one or more predicted performance indicator values satisfy one or more acceptability criteria, such as by exceeding some threshold value), to facilitate user selection of one or more particular candidate resins (e.g., resin lots).

Regardless of whether block 1430 is implemented automatically, manually, or with some combination thereof, block 1430 may include comparing the predicted performance indicator value(s) to a predetermined acceptability threshold. For example, block 1430 may include selecting a resin only if the corresponding performance indicator (e.g., HCP level) is below the acceptability threshold. Alternatively, if the multivariate statistical model outputs a range of values (e.g., the range for which a confidence threshold is exceeded), block 1430 may include comparing the range(s) of performance indicator values to a predetermined acceptability threshold. For example, block 1430 may include selecting a resin only if all values within the corresponding range (e.g., the corresponding range of HCP levels) are below the acceptability threshold.

At block 1440, the column chromatography purification process is performed using the resin that was selected (e.g., confirmed/approved) at block 1430 as the stationary phase in a column chromatography system. In some embodiments, the column chromatography system is a commercial-scale system. Block 1440 may be performed by the column chromatography system 140 of FIG. 1, for example, possibly with manual assistance. In multi-column systems, the selected resin may be used as the stationary phase in one, some or all of the columns.

In some embodiments, the method 1400 includes one or more other blocks not seen in FIG. 14. If the chromatography purification process/system from block 1440 is commercial-scale, for example, the method 1400 may include an additional block, prior to block 1410, in which the multivariate statistical model is trained using historical commercial-scale (and possibly also small-scale) chromatography purification data (i.e., historical inputs/feature values and corresponding performance indicators/labels).

Although the systems, methods, devices, and components thereof, have been described in terms of exemplary embodiments, they are not limited thereto. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent that would still fall within the scope of the claims defining the invention.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method of selecting raw materials for use in a column chromatography purification process, the method comprising:
for each of one or more candidate resins, receiving, by one or more processors of a computing system, a respective set of resin attribute values, the respective set of resin attribute values including at least one analytical measurement of the candidate resin;
for each of the one or more candidate resins, predicting, by the one or more processors applying the respective set of resin attribute values as inputs to a multivariate statistical model, a respective value of a performance indicator for the column chromatography purification process;
selecting a resin of the one or more candidate resins based at least in part on the one or more predicted respective values of the performance indicator; and
performing the column chromatography purification process using the selected resin as a stationary phase.

2. The method of claim 1, wherein predicting the respective value of the performance indicator further includes applying one or more harvest filtrate parameter values as inputs to the multivariate statistical model.

3. The method of claim 1, wherein predicting the respective value of the performance indicator further includes applying one or more purification process parameter values as inputs to the multivariate statistical model.

4. The method of claim 1, wherein predicting the respective value of the performance indicator includes applying (i) the respective set of resin attribute values, (ii) one or more harvest filtrate parameter values, and (iii) one or more purification process parameter values as inputs to the multivariate statistical model.

5. The method of claim 1, wherein predicting the respective value of the performance indicator includes predicting a level of host cell protein resulting from the column chromatography purification process.

6. The method of claim 1, wherein selecting the resin includes comparing the one or more respective values of the performance indicator to a predetermined acceptability threshold.

7. The method of claim 1, wherein:
predicting the respective value of the performance indicator includes predicting a respective range of values of the performance indicator; and
selecting the resin includes comparing each of the one or more respective ranges of values to a predetermined acceptability threshold.

8. The method of claim 1, wherein predicting the respective value of the performance indicator includes applying the respective set of resin characteristics as inputs to a projection on latent structures (PLS) regression model.

9. The method of claim 1, wherein, for each of the one or more candidate resins, receiving the respective set of resin attribute values includes receiving resin attribute values provided by a manufacturer or supplier of the candidate resin.

10. The method of claim 1, wherein:
the one or more candidate resins include a plurality of candidate resins that correspond to different manufacturing lots of a single resin type.

11. The method of claim 1, wherein:
the column chromatography purification process is a commercial-scale chromatography purification process; and
the method further comprises training the multivariate statistical model using historical small-scale and commercial-scale chromatography purification process data.

12. A system comprising:
a computing system that includes one or more processors and one or more memories, the one or more memories storing instructions that, when executed by the one or more processors, cause the computing system to, for each of one or more candidate resins
receive a respective set of resin attribute values, the respective set of resin attribute values including at least one analytical measurement of the candidate resin,
predict, by applying the respective set of resin attribute values as inputs to a multivariate statistical model, a respective value of a performance indicator for a column chromatography purification process, and
display the respective value of the performance indicator, or a result based on the respective value of the performance indicator, to a user to facilitate selection of a resin of the one or more candidate resins based at least in part on the one or more predicted respective values of the performance indicator; and
a column chromatography system configured to perform the column chromatography purification process using the selected resin as a stationary phase.

13. The system of claim 12, wherein predicting the respective value of the performance indicator further includes applying one or more harvest filtrate parameter values as inputs to the multivariate statistical model.

14. The system of claim 12, wherein predicting the respective value of the performance indicator further includes applying one or more purification process parameter values as inputs to the multivariate statistical model.

15. The system of claim 12, wherein predicting the respective value of the performance indicator includes applying (i) the respective set of resin attribute values, (ii) one or more harvest filtrate parameter values, and (iii) one or more purification process parameter values as inputs to the multivariate statistical model.

16. The system of claim 12, wherein the respective value of the performance indicator is a level of host cell protein resulting from the column chromatography purification process.

17. The system of claim 12, wherein predicting the respective value of the performance indicator includes predicting a respective range of values of the performance indicator.

18. The system of claim 12, wherein the multivariate statistical model includes a projection on latent structures (PLS) regression model.

19. The system of claim 12, wherein, for each of the one or more candidate resins, receiving the respective set of resin attribute values includes receiving resin attribute values provided by a manufacturer or supplier of the candidate resin.

20. The system of claim 12, wherein the one or more candidate resins include a plurality of candidate resins that correspond to different manufacturing lots of a single resin type.

21. A non-transitory computer-readable medium storing instructions that, when executed by one or more processors or a computing system, cause the computing system to, for each of one or more candidate resins:
receive a respective set of resin attribute values, the respective set of resin attribute values including at least one analytical measurement of the candidate resin;
predict, by applying the respective set of resin attribute values as inputs to a multivariate statistical model, a respective value of a performance indicator for a column chromatography purification process; and
display the respective value of the performance indicator, or a result based on the respective value of the performance indicator, to a user to facilitate selection of a resin of the one or more candidate resins, for use as a stationary phase in the column chromatography purification process, based at least in part on the one or more predicted respective values of the performance indicator.

22. The non-transitory computer-readable medium of claim 21, wherein predicting the respective value of the performance indicator further includes applying one or more harvest filtrate parameter values as inputs to the multivariate statistical model.

23. The non-transitory computer-readable medium of claim 21, wherein predicting the respective value of the performance indicator further includes applying one or more purification process parameter values as inputs to the multivariate statistical model.

24. The non-transitory computer-readable medium of claim 21, wherein predicting the respective value of the performance indicator includes applying (i) the respective set of resin attribute values, (ii) one or more harvest filtrate parameter values, and (iii) one or more purification process parameter values as inputs to the multivariate statistical model.

25. The non-transitory computer-readable medium of claim 21, wherein the respective value of the performance indicator is a level of host cell protein resulting from the column chromatography purification process.

26. The non-transitory computer-readable medium of claim 21, wherein the multivariate statistical model includes a projection on latent structures (PLS) regression model.
